(19) United States
(12) Patent Application Publication
(10) Pub. No.: US 2020/0069585 A1
(43) Pub. Date: Mar. 5, 2020

(54) COMPOSITIONS FOR UPREGULATING DEFENSIN EXPRESSION

(71) Applicant: KING SAUD UNIVERSITY, Riyadh (SA)

(72) Inventors: IBRAHIM S. ALNAJASHI, RIYADH (SA); GAMALELDIN I. HARISA, RIYADH (SA)

(21) Appl. No.: 16/120,120

(22) Filed: Aug. 31, 2018

(51) Int. Cl. A61K 9/127; A61K 31/201; A61K 9/107; A61K 47/28; A61K 47/24; A61K 47/44; A61K 9/00

(52) U.S. Cl. CPC A61K 9/127; A61K 31/201; A61K 9/1075; A61K 9/1277; A61K 47/24; A61K 47/44; A61K 9/0019; A61K 47/28

(57) ABSTRACT

Compositions for upregulating defensin expression include oleic acid and linoleic acid. The compositions can be in a liposomal, nanoliposomal, or nanoemulsion form. The compositions are useful in preventing, treating and ameliorating defensin-related conditions, such as preventing and treating HIV infection.

### BACKGROUND

### 1. Field

The disclosure of the present patent application relates to liposome, nanosome, and nanoemulsion compositions including oleic acid and compositions including oleic acid and linoleic acid.

### 2. Description of the Related Art

Human immunodeficiency virus (HIV) is a global pandemic, infecting about 36.7 million people world-wide. Half of those infected do not have access to antiretroviral therapy to treat their symptoms due to the high prices of such drugs, which can reach up to $900 (US Dollars) per month. Providing antiretroviral therapy to more patients can not only decrease the number of AIDS patients and AIDS related deaths, but also decrease the number of new cases and spreading rate by allowing those at risk to use such treatments as a prophylaxis to prevent the transmission of HIV in the first place.

HIV infects the immune system by binding to a cluster of differentiation-4 (CD4) proteins on T-cells, macrophages and dendritic cells, thereby gaining entry. HIV destroys infected immune cells by direct cell killing, pyroptosis and apoptosis, resulting in acquired immunodeficiency syndrome (AIDS). Despite a typical timeframe of about 9-11 years after HIV infection to development of AIDS, the life expectancy is around one year after development of AIDS. Mucosal tissues are the primary site of HIV transmission and replication. Many immune cells—i.e., lymphocytes, macrophages and dendritic cells—are scattered in mucosal tissues to support immunity. Thus, therapies targeting mucosal tissue immune defense hold promise as potential prophylactics or treatments for HIV/AIDS.

Mucosal tissues are supported by antimicrobial peptides, particularly, defensins. Defensins have been shown to act as protective agents against HIV infection (Quinones-Mateu et al., 2003; Sun et al., 2005), Herpes Simplex Virus (HSV) (Hazrati et al. 2006), Human papilloma virus (Buck et al., 2006), Acne Vulgaris bacteria (Catherine M. T et al, 2001), Candida Albican (Feng et al., 2005) and oral squamous cell carcinoma (Abiko et al., 1999). Moreover, defensins accelerate wound healing (Hirsch et al., 2008). Therefore, defensins are promising targets of action for discovery of antimicrobial and anticancer drugs.

Antiretroviral therapies (ARVTs) are commonly used in treatment of HIV. ARVTs are classified into different classes based on mode of action, including entry inhibitors, reverse transcriptase inhibitors, integrase inhibitors and protease inhibitors. ARVTs effectively decrease AIDS mortality rates. A combination of ARVTs known as highly active antiretroviral therapy (HAART) restores CD4 at early stages of HIV infection without complete eradication of HIV. During the HIV replication cycle, however, HIV produces new alleles that resist HAART. In this way, use of ARVT over a long duration induces drug resistance and failure of HIV viremia control. Moreover, HAART can cause lipid metabolism disorders, such as loss of subcutaneous fats and formation of fat deposits in the stomach and the neck. Additionally, HAART induces elevated blood triacylglycerols, cholesterol and insulin intolerance. HIV patients on antiretroviral therapy have shown elevated incidence of dyslipidemia, lipodystrophy and cardiovascular disease (CVD). Therefore, development of alternative anti-HIV therapies is still urgently needed, particularly antiviral strategies with low risk to the cardiovascular system, as chronically HIV-infected patients are vulnerable to CVD even aside from adverse effects of ARVT.

Essential fatty acids are present in natural oils, such as olive oil, and are known to have beneficial effects on the cardiovascular system. Accordingly, essential fatty acids have been used to minimize AIDS associated complications (Kozić Dokmanović et al., 2015). Moreover, fatty acids supplementation prevents antiretroviral therapy induced lipid disorders in patients with HIV/AIDS (Vieira, and Silveira, 2017). Auspiciously, fatty acids upregulate genes of host defense peptides (HDPs) (Sunkara el al., 2012). In addition, natural oils enriched with oleic acid have been reported to improve the function of the immune system (Sales-Campos et al., 2013). Diets enriched with fatty acids increase the survival of animals with AIDS (de Pablo et al., 2000). Mucosa of epithelial cells and phagocytes are enriched with HDPs, which act as a first line of defense against pathogens. HDPs activate different types of immune cells (Zeng et al., 2013). Mouse β-defensin-4 (mBD-4), a homolog of human β-defensin-2 (hBD-2), is upregulated by oleic acid treatment. In this context, free fatty acids show potential as antibacterial, antifungal and antiviral agents (Nakatsuji et al., 2010). This is attributed to HDPs killing a broad range of microbes including bacteria, fungi, parasites and enveloped viruses mainly through physical interaction and disruption of membranes. These pleiotropic effects make it beneficial to enhance synthesis of endogenous HDPs.

Relative to other drug delivery formulations, nanosomal drug delivery systems efficiently transport drugs to target sites (Harisa et al., 2017). As a result, nanosomal formulations such as nanoemulsions and nanoliposomes can enhance drug efficacy several fold. Such nanocarriers may be additionally tailored to protect molecules (Chopra et al., 2013). Liposomes have been successfully developed as fatty acid delivery systems and applied clinically and in effective therapeutic medications (Yang et al., 2009). As mentioned above, compounds that upregulate HDPs are attractive candidates for novel antiviral agents, and free fatty acids have long been known to possess a broad-spectrum antimicrobial activity.

Thus, compositions for upregulating defensins can be useful in treatment and/or prevention of HIV and AIDS while addressing the aforementioned problems and needs are desired.

### SUMMARY

Compositions for upregulating defensin expression prepared according to the present disclosure enhance defensing, e.g., beta-defensin, expression. As such, the compositions are useful in preventing, treating and ameliorating defensin-related conditions, such as preventing and treating HIV infection.

The compositions can include at least one of oleic acid and linoleic acid. The composition can be in liposomal, nanoliposomal, or nanoemulsion form. According to an embodiment, the compositions include a liposomal preparation including oleic acid and linoleic acid. According to an embodiment, the compositions include a nanoliposomal preparation including oleic acid and linoleic acid. According to an embodiment, the compositions include a nanoemulsion preparation including oleic acid and linoleic acid.

These and other features of the present disclosure will become readily apparent upon further review of the following specification and drawings.

### BRIEF DESCRIPTION OF THE DRAWINGS

FIG. 1 is a graph showing the effect of nanosomes on expression of mice beta defensin-4 gene compared to control mice after four hours of treatment according to the present teachings.

FIG. 2 is a graph showing the effect of nanosomes on expression of mice beta defensin-4 gene compared to control mice after twenty-four hours of treatment according to the present teachings.

### DETAILED DESCRIPTION OF THE PREFERRED EMBODIMENTS

Compositions for upregulating defensin expression prepared according to the present disclosure can enhance defensing, e.g., beta-defensin, expression. As such, the compositions according to the present teachings can be useful in preventing, treating and ameliorating defensin-related conditions, such as preventing and treating HIV infection.

The compositions can include at least one of oleic acid and linoleic acid in a liposomal, nanoliposomal, or nanoemulsion formulation. According to an embodiment, the composition includes a liposomal preparation including oleic acid and linoleic acid. According to an embodiment, the composition includes a nanoliposomal preparation including oleic acid and linoleic acid. According to an embodiment, the composition includes a nanoemulsion preparation including oleic acid and linoleic acid. When the nanosomal formulation comprises both oleic acid and linoleic acid, the oleic acid and linoleic acid are present in a 1:1 ratio. The compositions can be pharmaceutical compositions.

For a composition comprising an oleic acid and linoleic acid in nanoemulsion form, the average particle size ranges from about 1 nm to about 50 nm, e.g., from about 10 nm to about 20 nm. For a composition comprising an oleic acid and linoleic acid in the form of nanoliposomes, the nanoliposomes have an average particle size ranging from about 1 nm to about 200 nm, preferably around 150 nm.

A method of treating or preventing a defensin-related condition can include administering a therapeutically effective amount of a pharmaceutical composition including an oleic acid and a linoleic acid to a patient in need thereof. The pharmaceutical composition can be in a form selected from a nanoemulsion and a nanoliposome. The condition is preferably one that can be ameliorated by enhanced β-defensin peptide expression. For example, the condition can be selected from Human Immunodeficiency Virus (HIV), Acquired Immunodeficiency Syndrome (AIDS), Herpes simplex virus, Human papilloma virus, Acne Vulgaris bacteria, Candida Albican and oral squamous cell carcinoma. The composition can be administered by intraperitoneal injection or any other suitable manner. The present compositions are non-toxic and affordable (costing an estimated amount of about ten U.S. dollars monthly for treatment of the defensin-related condition).

The present compositions can induce secretion of beta-defensin proteins, which play a role in protecting the body from HIV. It is believed that beta-defensin expression is enhanced by inhibiting reverse transcriptase enzyme to stop the replication cycle at an early stage, or by inhibiting the two receptors (CCR5 and CXCR4) upon which the virus relies for the entry into the immune cell.

The present compositions can prevent HIV and/or AIDS. Post-exposure prophylaxis can include a short-term treatment with the present compositions which starts 72-hours within exposure to HIV. Prophylactic treatment using the present compositions can help prevent the virus from replicating in the body.

The present compositions can prevent complications of HIV, which leads to AIDS and many opportunistic infections.

An exemplary process for preparing a nanoliposome formulation of the composition can include forming a thin lipid film from a mixture of phospholipids, cholesterol, oleic acid and linoleic acid; hydrating the thin film with water to obtain multilamellar vesicles of liposomes; maintaining the multilamellar vesicles of liposomes at about 60° C. for about 2 h to anneal the bilayer structure; and reducing the liposome lamellarity by sonication.

An exemplary process for preparing a nanoemulsion formulation of the composition can include preparing an oily phase including oleic acid, linoleic acid, linseed oil, and soya bean oil, preparing an aqueous phase including a surfactant, a co-surfactant, and water; warming the aqueous phase and the oily phase; adding the aqueous phase drop-wise into the oily phase under stirring and homogenizing to form a course emulsion; and sonicating the course emulsion to produce a nanoemulsion.

As described herein, a “liposome” is a spherical vesicle composed of a unilamellar phase having at least one phospholipid bilayer. Liposomal vesicles that can be assembled inside aquatic milieu exhibit the phenomenon of hydrophilic and hydrophobic forces on phospholipid heads and tails. Hydrophobic tails face each other as shelter from water, whereas the hydrophilic heads face the water, thus forming multi-bilayers that give liposomes a vesicle shape. A liposome can entrap various compounds and can be used as a vehicle for delivery of pharmaceutical drugs. A “nanoliposome” is a liposome having a characteristic diameter less than 1 μm.

Also as described herein, an “emulsion” is a mixture of two or more liquids that are normally immiscible in which one liquid is dispersed in the other. In the present disclosure, a hydrophobic or “oily” phase liquid mixture is dispersed as droplets in an aqueous phase liquid mixture. A “nanoemulsion” is characterized by dispersed-phase droplets having a mean diameter less than 1 μm.

Compositions including oleic and linoleic fatty acids, as described herein, can be used for HIV and AIDS treatment. Free fatty acids (FFAs) influence fluidity of the plasma membrane, receptor and channel function, as well as act as signaling molecules affecting gene expression. FFAs are able to kill microorganisms directly as well as indirectly by upregulation of host defense peptide (HDPs) genes. FFAs are inducers of β-defensin-2 in mammalian cells. At the genetic level, the fatty acids have an ability to modulate histone deacetylase activities that play an important role in the control of gene expression. As described in the Examples below, compositions including oleic acid and linoleic acid in nanoliposomal form and in nanoemulsion form had a desirable particle size, zeta potential and polydispersity index. The compositions were administered to mice to test mBD-4 expression in the mice. The mBD-4 gene is homologous to beta-defensin in humans. The compositions effectively enhanced mBD-4 expression in mice.

The following examples illustrate the present teachings.

### EXAMPLES

### Materials and Method of the Exemplary Methods

1,2-dipalmitoyl-sn-glycero-3-phosphocholine (DPPC), soya bean oil and 1,2-distearoyl-sn-glycero-3-phosphoethanolamine-N-[amino(polyethylene glycol)-2000] (DSPE-PEG2000) were purchased from Lipoid GmbH (Ludwigshafen, Germany). Cholesterol (Chol), oleic acid, linoleic acid and linseed oil were purchased from Sigma-Aldrich (UK). Transcutol HP was obtained from Gattefosse (France). Chloroform (HPLC grade), methanol (HPLC grade) and tween 80 (Tw80) were obtained from Thermo Fisher Scientific (UK).

### Example 1

### Preparation of Nanosomes

### Nanoliposomal Preparation

Nanoliposomes were prepared by lipid film hydration technique (Doppalapudi et al., 2017). A lipid mixture was made of DPPC:Chol:DSPE-PEG2000:Tw80 (65:25:5:2 molar ratio) dissolved in 5 ml of chloroform/methanol (2:1 v/v). Oleic acid (10 mg/ml) or Oleic acid and linoleic acid (10 mg/ml, each) was added to the organic phase of the lipid mixture due to its high lipophilicity. The solvents were slowly removed on a Rotavapor® (Buechi, Flawil, Switzerland) maintained at 60° C. and at a speed of 100 rpm under vacuum for 1 h, allowing for formation of a thin film layer. The dried lipid film was hydrated with 5 ml of Milli-Q® water and multilamellar vesicles (MLVs) of liposomes were obtained and kept at 60° C. for 2 h to anneal the bilayer structure. Size reduction of liposome lamellarity was performed using a bath sonicator for a total duration of 30 min at 25° C. (see exemplary size distribution summarized in Table 1).

### Nanoemulsion Preparation

Nanoemulsions were prepared by hot homogenization followed by ultrasonication. Required amounts of all components were weighed and the oily phase and the aqueous phase were prepared separately. The oil phase included oleic acid and/or linoleic acid (10 mg/ml, each). The oily phase further included linseed oil (10% by volume) and soya bean oil (2% by volume). The aqueous phase included a mixture of surfactant and cosurfactant (4%)—wherein the surfactant and cosurfactant mixture was Tw80 and Transcutol at a 6:1 molar ratio—dissolved in water. The tonicity was adjusted by adding glycerol to the aqueous phase (a concentration of 2.25%, w/w). The oily and aqueous phases were warmed to 70° C. The aqueous phase was poured drop-wise into the oily phase slowly under stirring and then the combined solution was homogenized for about 5 min by a homogenizer to form a course emulsion. The coarse emulsion was then sonicated for about 20 min at 60% amplitude by a probe sonicator to produce a nanosome dispersion.

### Example 2

### Assays for Determining Nanosome Physical Properties and Bioactivity

### Nanosome Size Distribution Assay

The mean particle size and polydispersity index (PDI) of nanosomes obtained in the nanosome dispersions were determined by photon correlation spectroscopy using a Zetasizer Nano ZS (Malvern Instruments, Malvern, UK) at 25° C. The dispersion was diluted with deionized water before measurements to avoid multi-scattering phenomena. The zeta potential was assessed based on electrophoretic mobility taking the value with an average of 5 measurements for each prepared nanosome dispersion sample. All results presented in the subsequent exemplary results sections represent the average of triplicate measurements.

### Mouse Defensin-4 (MBD-4) Gene Expression Assay

18 male albino mice with body weight of 50±6.0 g, aged about 34 weeks were used as model organisms to assess effects of the nanosomes prepared as above. The mice were housed under conditions of controlled temperature (25±2° C.) with a 12:12-h day:night cycle, during which time they had free access to food and water ad libitum until the mice were acclimatized to laboratory conditions. The mice were maintained per national guidelines and protocols approved by the Institutional Animal Ethical Committee. The mice were fed ad libitum with a commercial diet for 5 days; the dietary components of chow were carbohydrates 72.2%, lipids 3.4%, proteins 19.8%, cellulose 3.6% vitamin and minerals 0.5% and salts 0.5% for 2 and lifted for 2 weeks for acclimatization. Afterward, mice were divided into three groups of 6 mice per group. Group 1 mice (control group) was administered normal saline. Group 2 mice was administered oleic acid and linoleic acid in a liposomal form. Group 3 mice was administered oleic acid and linoleic acid in a nanoemulsion form. Treatments were administered by intraperitoneal injection at a dose of 280 mg/kg. The average calculated dosage for all subjects was 0.6 ml/mouse for each nanosomal preparation.

Skin biopsies were taken from mice under light anesthesia at 4 hours and 24 hours after injection with nanosomes or control. Biopsies were collected from anesthetized mice according to the following protocol: skin to be biopsied was shaved and cleaned to remove hair and dirt residue. The shaved and cleaned skin to be biopsied was sterilized with iodine sterilizer, and then the biopsied skin tissue specimens were collected. Total RNA was extracted from the biopsied skin tissue specimens by TRIZOL (Invitrogen), according to the manufacturer's instructions. RNA concentrations were measured by spectrophotometry (NanoDrop, Thermo Fisher Scientific) and reported in μg/μl.

### RNA Expression Assay

RNA expression was studied by real-time PCR on cDNA prepared by reverse transcription, as follows.

cDNA molecules were synthesized using SuperScript™ VILO™ cDNA Synthesis Kit (Invitrogen). Reaction tubes were mixed gently and incubated at 25° C. for 10 minutes, followed by incubation at 42° C. for 60 minutes; reactions were terminated by incubation at 85° C. for 5 minutes. cDNA concentrations were measured by spectrophotometry (NanoDrop, Thermo Fisher Scientific) and tubes were kept at −20° C. until use.

Expression of mBD-4 gene was evaluated using GAPDH gene expression as a control. Real-time PCR was performed using TaqMan Gene Expression Master Mix (Invitrogen) and a Rotor Gene Q (QIAGEN) PCR machine. Thermal conditions were as follows: incubation at 50° C. for 2 minutes, followed by incubation at 95° C. for 10 minutes, then 40 cycles of a denaturation step at 95° C. for 15 seconds and an annealing/extension step at 60° C. for 1 minute.

### Exemplary Nanosome Physical Property and Bioactivity Results

Exemplary nanosomes prepared according to the present application were prepared having desirable particle size, zeta potential and polydispersity index, as shown in Table 1.

TABLE 1
Physicochemical characterization of oleic
acid and linoleic acid nanosomes.

| | | Oleic acid and | Oleic acid and |
|---|---|---|---|
| | Oleic acid | linoleic acid | linoleic acid |
| Codes | liposomes | liposomes | nanoemulsions |
| Particle size | 147.95 ± 5.02 | 151.13 ± 7.32 | 17.5 ± 2.68 |
| (nm) | | | |
| PDI | 0.182 ± 0.070 | 0.201 ± 0.0235 | 0.343 ± 0.0273 |
| Zeta potential | −9.16 ± 0.88 | −11.95 ± 0.78 | −21.45 ± 0.49 |
| (mV) | | | |

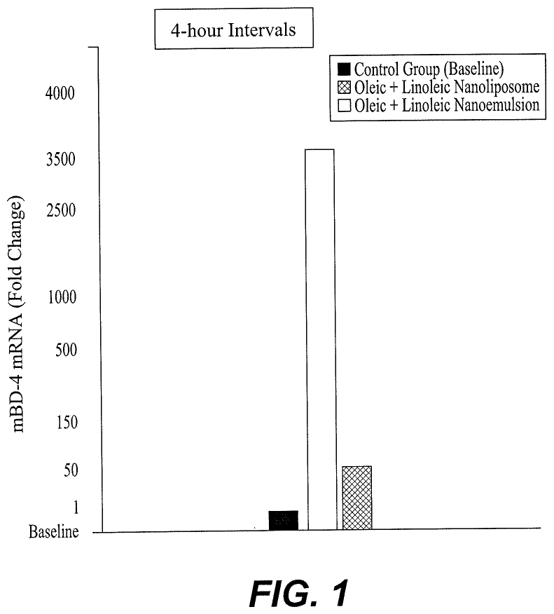

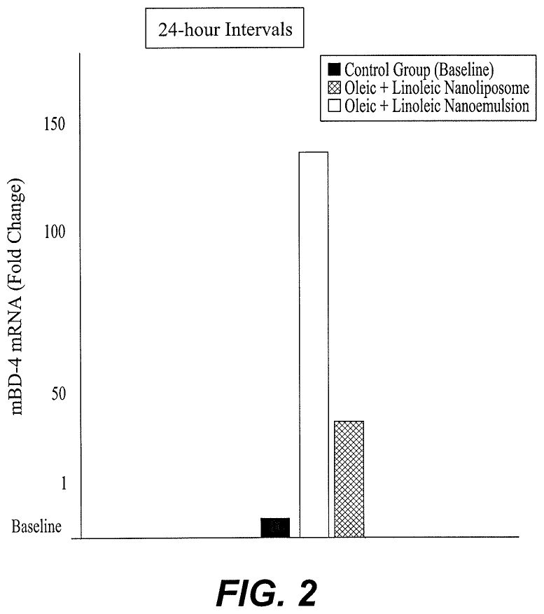

Exemplary nanosomes prepared as above were injected into the mice intraperitoneally, and mBD-4 mRNA expression of was studied at 4 and 24 hours after injection. At 4 hours after injection, a marked increase in mBD-4 mRNA expression was observed in mice administered the nanosomes compared to control. As indicated in FIG. 1, nanoliposomes including oleic acid and linoleic acid induced upregulation of mBD-4 gene 53-fold relative to control. However, nanoemulsions of oleic acid and linoleic acid induced upregulation of mBD-4 mRNA 3,666-fold compared to control. At 24 hours after injection, the mBD-4 mRNA expression under all conditions was decreased compared to respective 4-hour measurements; however, the nanoosome treated samples still exhibited higher expression relative to the control. Nanoliposome preparation elicited a 37 folds increase over control measured at 24 hours, while the nanoemulsion preparation elicited a 141 fold increase over control measured at 24 hours, as shown in FIG. 2. As such, the present nanosomes effectively enhance mBD-4 expression and provide an inexpensive and effective therapeutic agent for conditions affected by beta-defensin expression and activity.

It is to be understood that the present subject matter is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A pharmaceutical composition for upregulating defensin expression, comprising oleic acid and linoleic acid, the composition being in a form of a nanoliposome, wherein the nanoliposome has an average particle size of 150 nm.

2. (canceled)

3. The pharmaceutical composition according to claim 1, wherein the nanoliposome further includes phospholids and cholestrol.

4-5. (canceled)

6. The pharmaceutical composition according to claim 1, wherein a ratio of oleic acid to linoleic acid is 1:1.

7-15. (canceled)
